(19) United States
(12) Patent Application Publication
(10) Pub. No.: US 2024/0050030 A1
(43) Pub. Date: Feb. 15, 2024

(54) STANDARDISED METHOD FOR DETERMINING AN APNEA+HYPOPNEA INDEX OR A MARKER AS A FUNCTION OF SAID INDEX

(71) Applicants: UNIVERSITE GRENOBLE ALPES, Saint Martin D’Heres (FR); INSTITUT NATIONAL DE LA SANTE ET DE LA RECHERCHE MEDICALE, Paris (FR); CENTRE HOSPITALIER UNIVERSITAIRE GRENOBLE ALPES, Tronche (FR); UNIVERSITE DE BOURGOGNE, Dijon (FR)

(72) Inventors: Lionel SANCHEZ, Saint-Paul (FR); Fabrice MONNA, Gissey-Sur-Ouche (FR); Jean-Louis PEPIN, Grenobel (FR)

(21) Appl. No.: 18/259,033

(22) PCT Filed: Dec. 17, 2021
(86) PCT No.: PCT/EP2021/086651

§ 371 Date: Jun. 22, 2023

(30) Foreign Application Priority Data
Dec. 22, 2020 (FR) FR2013872

(51) Int. Cl. A61B 5/00; G16H 50/20

(52) U.S. Cl. CPC A61B 5/4818; G16H 50/20

(57) ABSTRACT

A process for determining a patient's apnoea+hypopnoea index or statis dependent on this index, including supplying a data set relating to a patient having, or making it possible to determine, characteristic data of the patient's maxillofacial morphology. The determination of the characteristic data is dependent on the positioning of at least four homologous points (1100, 1110), on a 3D scan of the head (60) of the patient (6). The process includes introducing this set into a machine learning model, trained to predict the apnoea+hypopnoea index, or the status, for the data set, from a database of different patients including maxillofacial morphology data, associated with an apnoea+hypopnoea index, or a status dependent on an apnoea+hypopnoea index, such that the model predicts an apnoea+hypopnoea index or a status for the data set relating to the patient.

### TECHNICAL FIELD OF THE INVENTION

The present invention relates to the field of the diagnosis of diseases using maxillofacial morphological characteristics with the assistance of image analysis machine learning. It finds a particularly advantageous application in the diagnosis of obstructive sleep apnoea syndrome or conditions for which sleep apnoea syndrome is very frequently associated.

### PRIOR ART

A form of sleep apnoea syndrome is associated with airway obstruction. When this obstruction is partial, the term hypopnoea is used. This obstruction may be complete, the term obstructive apnoea is then used.

Obstructive Sleep Apnoea-Hypopnoea Syndrome, also abbreviated to OSAHS, is a common condition in the population. The literature on the topic reports a prevalence of 4% among men and 2% among women (Young et al. The occurrence of sleep-disordered breathing among middle-aged adults, N Engl J Med, 328, 1230-1235, 1993), more particularly in adults over 50 years of age (Levy, et al. Obstructive sleep apnoea syndrome, Nature Reviews Disease Primers, 1, 15015, 2015). It consists of recurrent obstructions of the upper airways during sleep. OSAHS is associated with sleep fragmentation which is liable to promote daytime drowsiness, attention disorders, and increase the risk of accidents (Myers et al. Does this patient have obstruction sleep apnea? The rational clinical examination systematic review, JAMA, 310, 731-741, 2013). Intermittent hypoxia induced by this syndrome increases the prevalence of conditions such as cardiovascular and metabolic conditions, as well as patient mortality. As a general rule, this syndrome furthermore impairs the quality of life of patients and those close to them.

Following diagnosis, this syndrome is generally treated using a device used at night which maintains a continuous positive pressure which limits obstruction and stabilises the upper airways. There is a considerable need to screen for OSAHS among at-risk populations, representing millions of subjects, such as people suffering from high blood pressure, heart failure, or type 2 diabetes.

To diagnose this syndrome, the Berlin questionnaire is generally used by specialist practitioners, in order to establish a rapid diagnosis, which, combined with the specialist's experience, will refer the patient for more detailed investigations or not for the diagnosis of OSAHS. The Berlin questionnaire draws up an inventory of the patient's status in terms of snoring, drowsiness, and high blood pressure, then converted into a score making it possible to refer the patient for further investigation or not.

Further investigation consists of recording respiratory events during the patient's sleep. The reference investigation is a polysomnographic sleep study. This investigation is conducted in a hospital setting. Abnormal respiratory events (apnoea, hypopnoea) over the course of a night are quantified, in order to obtain an apnoea+hypopnoea index, which may also be referred to in the field as apnoea-hypopnoea index, or the acronym AHI. Polysomnography includes an electro-encephalogram, to assess sleep structure, and recording of cardiorespiratory events (nasal air flow rate, oxygen saturation of the blood, heart rate, chest and abdominal expansion).

Alternatively, ambulatory polygraphy is more accessible as it does not require hospitalisation. At the patient's home, a device records the air flow, oxygen saturation of the blood, respiratory events, snoring, and sleeping position overnight to obtain the apnoea+hypopnoea index.

However, even in the ambulatory version, these additional investigations cannot be prescribed systematically due to the high cost thereof, the small number of devices available and the shortage of qualified staff.

Therefore, there is a need to facilitate screening for OSAHS, particularly among at-risk populations, representing millions of subjects, such as people suffering from high blood pressure, heart failure, or type 2 diabetes.

Documents are available describing a link between morphological characteristics and OSAHS, particularly Banabilh, S. M., et al. Craniofacial obesity in patients with obstructive sleep apnea. Sleep Breath 13, 19-24 (2009). Some maxillofacial characteristics can be measured directly or indirectly, particularly measurements characterising craniofacial anatomy and increase in soft tissue volume (tongue, soft palate). Pharyngeal anatomy is recognised as one of the major factors in the onset of OSAHS. While a large proportion of these characteristics are visible, they remain nonetheless difficult to quantify and analyse, particularly for non-specialist practitioners.

One purpose of the present invention is therefore that of proposing to improve the reliability of and/or standardise, through the analysis of morphological characteristics of an upper part of the human body, the diagnosis of a condition, and more specifically the diagnosis of obstructive sleep apnoea-hypopnoea syndrome.

Other purposes, features and advantages of the present invention will become apparent upon reading the following description and examining the accompanying drawings. It is understood that other advantages can be incorporated therein.

### SUMMARY OF THE INVENTION

To achieve this aim, according to a first aspect, a process is provided for determining a patient's apnoea+hypopnoea index or a status dependent on an apnoea+hypopnoea index, comprising:
supplying a data set relating to a patient, to a remote server or a computer program product. The data set relating to the patient comprises, or makes it possible to determine, characteristic data of the at least maxillofacial morphology of the patient with respect to a reference morphology. Said characteristic data are dependent on the positioning of at least four homologous points, on a 3D scan of a portion of the patient's head representing their at least maxillofacial morphology. Where applicable, the determination of said characteristic data is dependent on the positioning of at least four homologous points, on a 3D scan of a portion of the patient's head representing their at least maxillofacial morphology,
introducing the data set relating to the patient, comprising the characteristic data of the patient's at least maxillofacial morphology, into a machine learning model trained to predict an apnoea+hypopnoea index, or a status dependent on an apnoea+hypopnoea index, for said data set, from a plurality of data sets of a database relating to a set of separate patients, each data set of the database:
   comprising at least maxillofacial morphology data relating to a patient from the set, and
   being associated with an apnoea+hypopnoea index, or a status dependent on an apnoea+hypopnoea index,
      such that the learning model predicts an apnoea+hypopnoea index, or a label dependent on an apnoea+hypopnoea index, for the data set relating to the patient.

Thus, the patient's morphological characteristics are compared in an automated fashion with the database, to obtain by comparison the apnoea+hypopnoea index or the status dependent on this index. The homologous points make it possible to improve the reliability of the alignment between the characteristic data of the patient's morphology and those of the set of patients of the database, and thus improve the reliability of prediction. This process therefore makes it possible to avoid the observation of these characteristics by a specialist practitioner, and the variability that can arise from this observation, for example from one practitioner to another.

The characteristic data of the patient's at least maxillofacial morphology being obtained from a 3D scan comprising a large number of items of information, positioning disparities can cause substantial variability in results and therefore limit the reliability of the process. The reference morphology corresponds to that of a reference individual and makes it possible to form a reference grid to be applied to the patient's 3D scan in order to limit non-relevant variability of the data. The 3D scan is thus processed so as to align the homologous points of the patient and those of the grid. The patient's characteristic data based on the positioning of the homologous points with respect to this reference morphology are therefore obtained by minimising deviations not linked with apnoea-hypopnoea syndrome. The reliability of the process is therefore improved.

Obtaining the apnoea+hypopnoea index, or the status dependent on this index, furthermore does not require, for the patient to be diagnosed, the recording of respiratory events during their sleep. Performing polysomnography or polygraphy is avoided for the patient to be diagnosed, with the constraints caused thereby, such as difficulties falling asleep, having to travel multiple times. Furthermore, interpreting polysomnography and polygraphy results is complex and must be performed by a specialist practitioner, to arrive at the apnoea+hypopnoea index.

The process therefore makes it possible to standardise and facilitate, based on a patient's morphological characteristics, obtaining the apnoea+hypopnoea index, or the status dependent on this index, and therefore the diagnosis of a condition linked with the apnoea+hypopnoea index, for example the diagnosis of OSAHS. Comfort and ease of access to care for the patient are furthermore improved with respect to existing solutions. Obtaining the apnoea+hypopnoea index, or the status dependent on this index, is simplified and accelerated with respect to existing solutions, since this index is obtained based on a 3D scan of the patient. The process can be implemented by a practitioner specialised in the condition or not, or by any operator such as a healthcare professional, for example a pharmacist. Its result can furthermore be returned quickly, for example from the consultation, the apnoea+hypopnoea index or status being obtained via the machine learning model.

According to an embodiment, the status is more specifically a qualitative or quantitative value of risk of the patient having the condition. The status can have at least two values, one value associated with a low risk and at least one value associated with an established risk. Preferably, the status has, from the at least one value associated with an established risk, at least one value associated with a moderate established risk of having the condition, and at least one value associated with a severe established risk of having the condition.

The status is dependent on the apnoea-hypopnoea index, and particularly dependent on the standard apnoea+hypopnoea index value for a condition, for example OSAHS. For example, the status has:
a low risk value corresponding to an apnoea+hypopnoea index value substantially less than 15,
an established risk value corresponding to an apnoea+hypopnoea index value substantially greater than 15.

The status can furthermore have:
a moderate established risk value corresponding to an apnoea+hypopnoea index value substantially between 15 and 30,
a severe established risk value corresponding to an apnoea+hypopnoea index value substantially greater than 30.

When the predicted apnoea+hypopnoea index for the data set relating to the patient is greater than a standard apnoea+hypopnoea index threshold value, and more specifically greater than 15, or, when the predicted status has at least one value associated with established risk, the process can comprise the diagnosis of a condition of the patient, for example OSAHS. The process according to the first aspect can be a process for diagnosing the condition, and has the advantages described above. The process thus makes it possible to improve the reliability of and standardise, through the analysis of morphological characteristics of an upper part of the human body, the diagnosis of a condition, and more specifically the diagnosis of obstructive sleep apnoea-hypopnoea syndrome.

According to an example, the process can comprise:
a comparison of the predicted apnoea+hypopnoea index for the data set relating to the patient with a standard apnoea+hypopnoea index threshold value,
an observation that the predicted apnoea+hypopnoea index for the data set relating to the patient is greater than the standard apnoea+hypopnoea index threshold value, for example 15,
a diagnosis of a condition linked with the apnoea+hypopnoea index, for example OSAHS.

According to an example, the process can comprise:
an observation that the status has an established risk value of having a condition linked with the apnoea+hypopnoea index
a diagnosis of the condition linked with the apnoea+hypopnoea index, for example OSAHS.

According to an example, when the predicted apnoea+hypopnoea index for the data set relating to the patient is substantially between 15 and 30, or when the predicted status has a value associated with moderate established risk, the patient is diagnosed with a moderate degree of obstructive sleep apnoea-hypopnoea syndrome, and when the predicted apnoea+hypopnoea index for the data set relating to the patient is substantially greater than 30, or when the predicted status has a value associated with severe established risk, the patient is diagnosed with a severe degree of obstructive sleep apnoea-hypopnoea syndrome. Thus, the process makes it possible to determine a degree of OSAHS severity.

A second aspect, that can be combined with or separated from the first aspect, relates to a method executed by a computer and comprising at least the steps listed above relative to the process according to the first aspect. A method executed by a computer can comprise any step of the process that can be executed on a computer, for example by a computer program product and/or a remote server. The process for determining the patient's apnoea+hypopnoea index can more specifically comprise the method executed by a computer according to this aspect.

A third aspect relates to a remote server capable of communicating with a computer program product, and comprising means for implementing the process according to the first aspect and/or the method according to the second aspect. The remote server comprises:
a database relating to a set of distinct patients comprising a plurality of data sets, each data set of the database:
   comprising at least maxillofacial morphology data relating to a patient from the set,
   being associated with an apnoea+hypopnoea index, or a status dependent on an apnoea+hypopnoea index, and
a machine learning model trained to predict an apnoea+hypopnoea index, or a status dependent on an apnoea+hypopnoea index, for said data set, from the database.

The remote server is configured to:
receive the data set relating to the patient comprising, or making it possible to determine, characteristic data of the patient's at least maxillofacial morphology with respect to a reference morphology, where applicable the determination of said data being dependent on the positioning of at least four homologous points, on a 3D scan of a portion of the patient's head representing their at least maxillofacial morphology,
introduce the data set relating to the patient, comprising the characteristic data of the patients at least maxillofacial morphology, in the trained machine learning model, such that the learning model predicts an apnoea+hypopnoea index or a status dependent on an apnoea+hypopnoea index, for the data set relating to the patient.

According to an example, the remote server can be configured to update the database following a receipt of a dataset relating to the patient, supplemented by the patient's apnoea+hypopnoea index measured by polysomnography or ambulatory polygraphy. Thus, if the patient undergoes polysomnography or ambulatory polygraphy, for example following the diagnosis of the condition by the process according to the first aspect, the apnoea+hypopnoea index measured can be included in the data set relating to the patient. This set can be added to the database and thus update it, to improve the machine learning model.

A fourth aspect relates to a computer program product comprising instructions, which when they are performed by at least one processor, executes at least, of the process according to the first aspect and/or the method according to the second aspect, sending to the remote server the data set relating to the patient, the data set relating to the patient comprising, or making it possible to determine, characteristic data of the patient's at least maxillofacial morphology with respect to a reference morphology, said characteristic data being dependent on the positioning of at least four homologous points, on a 3D scan of a portion of the patient's head representing their at least maxillofacial morphology, where applicable the determination of said data being dependent on the positioning of at least four homologous points, on a 3D scan of the patient's head representing their at least maxillofacial morphology.

According to an example, the computer program product comprises the determination of said characteristic data according to the positioning of at least four homologous points, on the 3D scan of a portion of the patient's head representing their at least maxillofacial morphology.

According to an example, the computer program product can be configured to receive, from the remote server, the apnoea+hypopnoea index.

A fifth aspect relates to a computer program product comprising instructions, which when they are performed by at least one processor, executes at least, of the process according to the first aspect and/or the method according to the second aspect:
reception of the data set relating to the patient, the data set relating to the patient comprising, or making it possible to determine, characteristic data of the patient's at least maxillofacial morphology with respect to a reference morphology, said characteristic data being dependent on the positioning of at least four homologous points, on a 3D scan of a portion of the patient's head representing their at least maxillofacial morphology, where applicable the determination of said data being dependent on the positioning of at least four homologous points, on a 3D scan of a portion of the patient's head representing their at least maxillofacial morphology,
introduction of the data set relating to the patient, comprising the characteristic data of the at least maxillofacial morphology of the patient, into the machine learning model trained to predict an apnoea+hypopnoea index, or a status dependent on an apnoea+hypopnoea index, for said data set, from a plurality of data sets of a database relating to a set of separate patients, each data set of the database:
   comprising at least maxillofacial morphology data relating to a patient from the set, and
   being associated with an apnoea+hypopnoea index, or a status dependent on an apnoea+hypopnoea index,
      such that the learning model predicts (14) an apnoea+hypopnoea index, or a status dependent on an apnoea+hypopnoea index, for the data set relating to the patient.

A sixth aspect relates to a kit for implementing the process according to the first aspect, comprising at least one computer program product according to any one of the preceding two claims, a scanner capable of the acquisition of a 3D scan of a portion of the patients head representing their at least maxillofacial, and preferably maxillofacial and submandibular, morphology. The kit can furthermore comprise a device for aligning the patient's head.

### BRIEF DESCRIPTION OF THE FIGURES

The aims, purposes, characteristics and advantages of the invention will be better understood upon reading the detailed description of one embodiment thereof, which is illustrated by means of the following accompanying drawings, in which:

FIG. 1 represents the steps of the process for determining a patient's apnoea+hypopnoea index, according to an embodiment example, where optional steps of the process are indicated with dotted lines and parallel paths indicate variants of the process.

FIG. 2 represents the kit and the remote server, according to an embodiment example.

FIG. 3 illustrates the verification of the alignment of the patient's head by an alignment device, according to an embodiment example.

FIG. 4 represents a characteristic data of a patient's maxillofacial and submandibular morphology, determined from seven homologous points.

FIG. 5 is a graph of the proportion of variance expressed by the characteristic data of the morphology represented in FIG. 4, according to an increasing number of principal components, after a principal component analysis.

FIGS. 6A and 6B represent an example of projection of the patient (black), measured several times, and of patients of the database (white) in a plane defined respectively by the first and the second principal components in FIG. 6A; the first and the third principal components in FIG. 6B.

The drawings are provided by way of example and are not intended to limit the scope of the invention. They constitute diagrammatic views intended to ease the understanding of the invention and are not necessarily to the scale of practical applications.

### DETAILED DESCRIPTION OF THE INVENTION

Before giving a detailed review of embodiments of the invention, optional features are set out below, which can be used as an alternative to or in combination with one another:
the data set relating to the patient can be supplied in the form of:
   a 3D scan of a portion of the patient's head representing the patient's at least maxillofacial, and preferably maxillofacial and submandibular, morphology,
   a 3D scan of a portion of the patient's head representing the patient's at least maxillofacial, and preferably maxillofacial and submandibular, morphology, wherein homologous points are positioned
   characteristic data of the patients morphology, determined according to the positioning of homologous points on a 3D scan representing the patient's at least maxillofacial, and preferably maxillofacial and submandibular, morphology,
the supplied data set relating to the patient comprising a 3D scan of a portion of the patient's head representing the patient's at least maxillofacial morphology, the process comprises, prior to the introduction of the data set relating to the patient in the machine learning model:
   positioning the at least one homologous points on the 3D scan,
   determining, from the homologous points positioned, characteristic data of the patient's at least maxillofacial morphology with respect to the reference morphology.
the supplied data set relating to the patient comprising a 3D scan of a portion of the patient's head, whereon the at least four homologous points are positioned, the process comprises, prior to the introduction of the data set relating to the patient in the machine learning model, determining, from the at least four homologous points positioned, characteristic data of the patient's at least maxillofacial morphology with respect to the reference morphology,
the supplied data set relating to the patient comprises characteristic data of the patient's at least maxillofacial morphology with respect to the reference morphology, as determined according to the positioning of the homologous points on a 3D scan of a portion of the patient's head,
the data set relating to the patient furthermore comprises, or makes it possible to furthermore determine, characteristic data of the patient's submandibular morphology with respect to the reference morphology, said characteristic data being dependent on the positioning of at least three supplementary homologous points, on the 3D scan of a portion of the patient's head representing their at least maxillofacial and submandibular morphology, where applicable the determination of said data being dependent on the positioning of at least three supplementary homologous points, on the 3D scan of a portion of the patient's head representing their maxillofacial and submandibular morphology,
the three supplementary homologous points corresponding to locations on the patient's chest, for example the three homologous points corresponding to the shoulder joints, and/or the suprasternal notch.
determining the characteristic data of the patient's at least maxillofacial, preferably maxillofacial and submandibular, morphology with respect to the reference morphology comprises a reduction of the dimensionality of the data from the positioning of the at least four homologous points, preferably the at least seven homologous points, for example by a principal component analysis,
the data set relating to the patient furthermore comprises additional clinical information data on the patient. These data are for example chosen from a patient body mass index, clinical examination upper airway obstruction indexes, medical questionnaire data. The machine learning model can be trained to predict the apnoea+hypopnoea index, or the status dependent on an apnoea+hypopnoea index, for the data set relating to the patient, from the plurality of data sets of the database, each data set of the database comprising said additional clinical information data relating to a patient of the set,
the data set relating to the patient furthermore comprises, or makes it possible to furthermore determine, supplementary characteristic data of the patient's maxillofacial morphology in at least one position from a prognathic position and a retrognathic position, preferably a maximum prognathic position and a maximum retrognathic position,
the process comprises the acquisition of a 3D scan of a portion of the patient's head representing their at least maxillofacial, preferably maxillofacial and submandibular, morphology,
the acquisition of the 3D scan comprises a verification of the alignment of the patient's head, and more specifically of the horizontal position of the reference plane of the patient's head, by an alignment device,
when the data set relating to the patient is supplied to a remote server, the process comprises returning, from the remote server to a computer program product, the apnoea+hypopnoea index, or the status, predicted for the data set relating to the patient,
when the data set relating to the patient is supplied to a computer program product or when a computer program product receives the apnoea+hypopnoea index, or the status, returned from the remote server, the process comprises displaying, by the computer program product, the apnoea+hypopnoea index, or the status, predicted for the data set relating to the patient,
determining the characteristic data of the patient's at least maxillofacial morphology with respect to the reference morphology comprises:
   applying a reference grid from the reference morphology to the patient's 3D scan,
   positioning the homologous points on the patient's 3D scan,
   aligning the patient's homologous points and the reference grid,
the reference grid comprises semi-homologous points disposed between the homologous points, preferably equidistant from each other,
determining the patient's characteristic data comprises an iterative adjustment of the semi-homologous points on the reference grid so as to minimise the deformation of the grid with respect to the patient's 3D scan,
the characteristic data of the patient's at least maxillofacial morphology with respect to the reference morphology comprise a set of homologous points and a set of semi-homologous points disposed between the homologous points, preferably the set of semi-homologous points comprises at least 200 and preferably at least 300 points. Thus, the data comprise a large number of points to represent the patient's morphology more precisely. The reliability of the process is thus improved.

It is specified that, within the scope of the present invention, the term 3D scan (three-dimensional scan) refers to an image resulting from a procedure supplying the three-dimensional image obtained from at least one medical imaging technique. Preferably, the medical imaging technique is non-irradiating for the patient. One type of medical imaging envisaged is infrared light scanning. This technique has the advantage of being safe and non-ionising for the patient. Note that it can be envisaged to obtain a 3D scan by photogrammetry, which is equally safe for the patient. It can furthermore be envisaged to obtain a 3D scan by lasergrammetry. Within the scope of the present invention, the 3D scan is performed in the frontal position with respect to the patient and represents, around an axis of revolution parallel with the vertical or longitudinal axis of the patient, at least one third, and preferably half, of their maxillofacial morphology, and preferably their maxillofacial and submandibular morphology. According to an example, the 3D scan represents the frontal morphology of the patient's head:
from the eyes to the chin, and to the ears, for the maxillofacial morphology,
from the eyes to the chest, and more specifically to the shoulder joints and the suprasternal notch, and to the ears, for the maxillofacial and submandibular morphology.

It can be envisaged that the 3D scan comprises a plurality of scans, for example a scan of the maxillofacial morphology and a separate scan of the submandibular morphology. A plurality of two-dimensional scans can furthermore be used to form the 3D scan.

The term “homologous points” denotes points representing the same location of the face or chest between different 3D scans, for example of separate patients. These points represent a homology between 3D scans, i.e. the same morphological characteristic. By way of homologous point, mention can be made of:
the junction between the earlobe and the cheeks,
the nasal base,
one, and preferably both, pupil(s),
one, and preferably both, shoulder joint(s),
the suprasternal notch.

The term “clinical information on the patient” denotes data relating to a patient acquired by an investigation by a healthcare professional. This information can comprise at least one of the following items: patient's age, sex, height, weight, BMI, anthropological type, health status, medical history, responses to a health questionnaire, clinical investigation data and risk factors.

The term “machine learning” refers to one or more computing algorithms capable of automatically performing one or more predictions and/or classifications without explicit programming. The computing algorithm can construct the learning model from learning data, and particularly from the database. Note that the machine learning model can be linked with the database hosted by the remote server. According to an example, the learning model is itself hosted by the remote server. The machine learning model may not be linked with the database hosted by the remote server, during the implementation of the process. For example, the machine learning model was trained on the learning data, then was implemented in a computer program product capable of operating independently from the server, during the implementation of the process. This does not exclude updates of the machine learning model, for example by communication between the remote server and the computer program product.

It is specified that within the scope of the present invention, a parameter that is “substantially equal to/greater than/less than” a given value is understood to mean that this parameter is equal to/greater than/less than the given value, to within more or less 10%, or even to within more or less 5%, of this value.

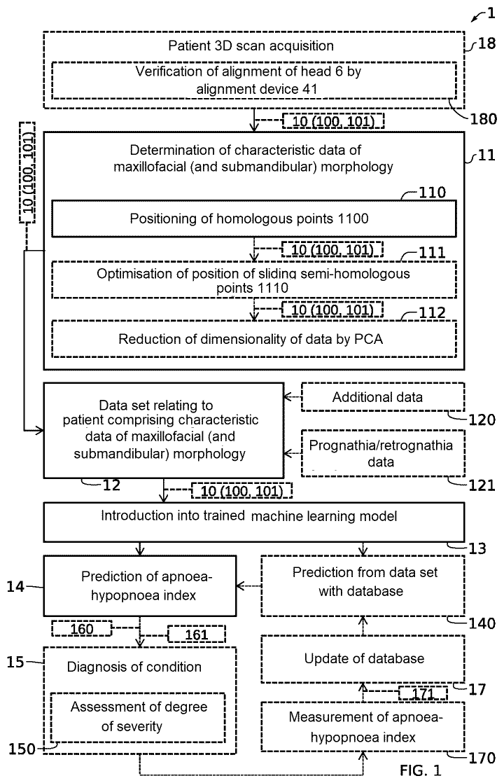

The process 1 for determining a patients apnoea+hypopnoea index, according to an embodiment example, is now described with reference to FIG. 1.

The process 1 comprises the supply 10 of a data set relating to a patient. The data set relating to the patient comprises, or makes it possible to determine, characteristic data of the patient's at least maxillofacial, and preferably maxillofacial and submandibular, morphology with respect to a reference morphology. These data are determined from a 3D scan of the patient's head 6, representing their maxillofacial morphology, and preferably their maxillofacial and submandibular morphology.

The 3D scan be processed to determine 11 characteristic data of the patient's morphology. Hereinafter, these data are considered in a non-limiting manner to be characteristic of the patient's maxillofacial and submandibular morphology. Unless specified otherwise, the features described hereinafter can be applied to the example whereby the characteristic data are determined from a 3D scan representing only the patient's maxillofacial morphology.

The data set 12 relating to the patient, comprising these characteristic data of the morphology, is introduced 13 into the machine learning model. The machine learning model is trained to predict, from a database, an apnoea+hypopnoea index and/or a status dependent on this index, for the data set 12 relating to the patient.

The database comprises a plurality of data sets of separate individuals, hereinafter referred to as patients. These patients can be healthy or have a condition linked with the apnoea+hypopnoea index. Each data set comprises at least morphology data and the apnoea+hypopnoea index, and/or a status dependent on this index, of a patient.

The apnoea+hypopnoea index can more specifically have been measured, for example by polygraphy or preferably by polysomnography. The status can be obtained from a conversion of an apnoea+hypopnoea index into a qualitative or quantitative risk value of the patient having a condition. This conversion can for example be performed when compiling the database, or when updating this database, described hereinafter. In particular, the status can have a value associated with a low risk and at least one value associated with an established risk. Preferably, the status has at least one value associated with a moderate established risk of having the condition, and at least one value associated with a severe established risk of having the condition. For this, the status can be obtained from the apnoea+hypopnoea index, with respect to one or more standard threshold values of this index. These threshold values are preferably standards of risk of having a condition, established in the medical field for the condition in question.

For OSAHS or linked conditions, the condition can have:
a low risk value corresponding to an apnoea+hypopnoea index value substantially less than 15,
an established risk value corresponding to an apnoea+hypopnoea index value substantially greater than 15.

The status can furthermore have:
a moderate established risk value corresponding to an apnoea+hypopnoea index value substantially between 15 and 30,
a severe established risk value corresponding to an apnoea+hypopnoea index value substantially greater than 30.

The machine learning model is a supervised training model trained to predict in an automated manner the apnoea+hypopnoea index and/or the status for the data set 12 relating to the patient. The supervised learning model can particularly comprise a mathematical model in which the input data are the data set 12 relating to the patient, and the output data item is the apnoea+hypopnoea index and/or the status.

The database comprising a plurality of patient data sets, each comprising the apnoea+hypopnoea index, and/or a status dependent on this index, and the learning model having been trained on this database, it is understood that the learning model is trained to classify the data set 12 relating to the patient to assign them the status, and/or to predict, from the data set 12 relating to the patient, the apnoea+hypopnoea index. When a status is predicted 14, the learning model can perform a binary, or ternary or greater classification according to the number of possible status values. When the apnoea+hypopnoea index is predicted 14, the learning model can perform a regression. Intermediately, the learning model can classify the individuals in ordered groups.

As such, the process 1 makes it possible to obtain in a standardised and automated manner the patient's apnoea+hypopnoea index, from their maxillofacial and submandibular morphology. The apnoea+hypopnoea indexes are measured using standard clinical methods for the patients of the database. It is, however, not necessary for this measurement to be made for the patient for whom the apnoea+hypopnoea index is sought. Indeed, the characteristic morphology data are determined from a 3D scan of the patient's head 6. The process 1 therefore makes it possible to avoid measurement by polygraphy or polysomnography, for this patient. The process 1 furthermore makes it possible to avoid an observation of the patient's morphology by a specialist practitioner, and/or at least minimise, or prevent, variability or a lack of assessment capable of arising from this observation.

From the apnoea+hypopnoea index or the status predicted for the patient, a diagnosis 15 of a condition linked with this index can be established, preferably supplemented by an assessment 150 of the degree of severity of the condition. The condition linked with this index can be OSAHS, and more specifically Obstructive Sleep Apnoea Syndrome, commonly abbreviated to OSAS. In other conditions, OSAHS is a very prevalent condition and it is preferable to diagnose it to establish the prognosis of the other condition, and furthermore even better control it. By way of non-limiting examples, this applies particularly for diabetes and hypertension. In some rare conditions affecting maxillofacial morphology, OSAHS is very common and the process 1 would make it possible to facilitate their characterisation and/or their diagnosis. By way of non-limiting example, this applies particularly for acromegaly, Crouzon's disease, Apert syndrome, Down syndrome.

The process can therefore be used to diagnose a condition for which OSAHS is prevalent and/or for which OSAHS is common. These conditions can be referenced by ICD-10, the international disease classification, particularly classifying diseases, signs, symptoms, social circumstances and external causes of diseases or injuries, and published by the World Health Organization:
J47-3: essentially comprising sleep apnoea-hypopnoea,
110 comprising hypertension,
148 comprising atrial fibrillation and flutter,
1219 comprising myocardial infarction,
E119 comprising type 2 diabetes,
K758 NASH (Nonalcoholic Steatohepatitis),
1509 comprising heart failure,
E220 comprising acromegaly,
Q901 comprising Down syndrome.

For this, the apnoea+hypopnoea index predicted for the patient can be compared to one or more standard values differentiating:
a healthy person from a person suffering from the condition,
different degrees of severity of the condition.

Alternatively or additionally, the status predicted for the patient can be observed to diagnose the condition, preferably according to the degree of severity of the condition.

Note that it can be envisaged to establish the diagnosis on the basis of the predicted apnoea+hypopnoea index, or status, for example supplemented with clinical information on the patient, particularly when the apnoea+hypopnoea index is substantially around the standard values above, and the diagnostic probability is reinforced by the symptoms and/or the context.

According to an example, for the diagnosis of OSAHS and/or conditions linked with OSAHS, for example the conditions cited above, the apnoea+hypopnoea index predicted for the patient can be compared to a threshold value of 15, differentiating a healthy person for an index substantially less than 15, from a person with OSAHS for an index substantially greater than 15. The apnoea+hypopnoea index predicted for the patient can be compared to the threshold value of 30 differentiating a person with a moderate degree of OSAHS for an index substantially between 15 and 30, and a person with a severe degree of OSAHS for an index substantially greater than 30. Hereinafter, the process is considered in a non-limiting manner to make it possible to predict the apnoea+hypopnoea index for the patient. The following features however also apply to the embodiment whereby a status is predicted.

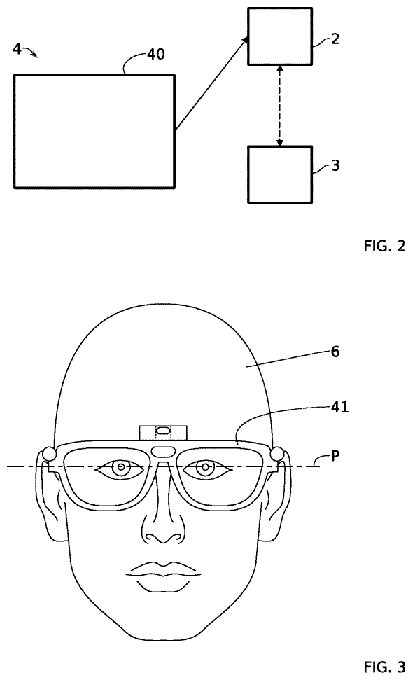

The process 1 can be implemented by a computer program product 2 and/or a remote server 3. The computer program product 2 and/or the remote server 3 can be configured to implement any step executed by computer of the process 1, particularly according to the communication scenario thereof. Different communication scenarios can be envisaged, for example illustrated by FIGS. 1 and 2. According to an example, the data set relating to the patient can be supplied 10 to a computer program product 2 comprising the machine learning model. For example, the data set is supplied 10 by an operator, for example a healthcare professional, in software. The computer program product 2 can be configured to process the 3D scan, or data from this scan, to determine 11 the characteristic data of the patient's morphology. The computer program product 2 can be configured to introduce 13 the data set 12 relating to the patient, comprising characteristic data of the patient's morphology, in the machine learning model. The machine learning model being trained, it is not necessary for the computer program product to comprise the database. Once identified 14, the apnoea+hypopnoea index predicted for the patient can then be displayed 161 by the computer program product 2.

According to another example, the data set relating to the patient can be supplied 10 to a remote server 3, for example via a computer program product 2. For example, the data set is supplied 10 by an operator in software, which then sends 101 the data set to be received 100 by the remote server 3. The computer program product 2 can be configured to process the 3D scan, or data from this scan, to determine 11 the characteristic data of the patient's morphology, prior to sending 101 to the remote server 3. The remote server 3 can be configured to process the 3D scan, or data from this scan, to determine 11 the characteristic data of the patient's morphology, following the receipt 100 of the data set. The remote server 3 can be configured to introduce 13 the data set 12 relating to the patient, comprising characteristic data of the patient's morphology, in the machine learning model. The remote server 3 hosting the database, the machine learning model can compare 140, to the database, the data set introduced, to predict 14 the apnoea+hypopnoea index. The apnoea+hypopnoea index can then be returned 160 by the remote server 3 to the computer program product 2, for example for the display 161 thereof.

It is understood that the different steps of the process can be performed by one and/or the other of the computer program product 2 and the remote server 3, according to the communication scenario between these elements.

The data set relating to the patient can be supplied 10 in the form of:
a 3D scan representing the patient's morphology
a 3D scan representing the patient's morphology, wherein homologous points are positioned, described in more detail hereinafter,
characteristic data of the patient's morphology, determined 11 according to the positioning of homologous points on a 3D scan representing the patient's morphology, as described in more detail hereinafter.

According to the form of the data 10, the computer program product 2 and/or the remote server 3 can be configured to perform the steps resulting in the determination 11 of the characteristic data of the patient's morphology. The computer program product 2 and/or the remote server 3 can furthermore be configured to compile the data set 12 relating to the patient comprising the characteristic data of their morphology. The different steps that the process 1 can comprise are now detailed, with reference to FIG. 1.

The process can comprise the acquisition 18 of the scan. For this, the 3D scan can be performed by a scanner 40, for example illustrated in FIG. 2. This scanner 40 can be part of a kit 4, communicating with the computer program product 2, itself capable, where applicable, of communicating with the remote server 3, such as for example illustrated by FIG. 2. The scan can typically be performed approximately 40 to 50 cm from the patient's head 6. The scanner 40 can project a cloud of infrared light. For example, a System Sense© commercial 3D scanner can be used. A person skilled in the art will be able to envisage any technical medical imaging means for acquiring the 3D scan.

Preferably, the patient's posture is natural, with the mouth closed, the lips in a neutral expression, and the eyes looking towards the horizon. In order to facilitate the determination 11 of the characteristic data of the patient's morphology, the process can comprise a verification 180 of the alignment of the patients head 6, and more specifically a verification of the horizontal position of the reference plane of their head 6. The reference plane is a plane passing through the upper portion of the connection between the ears and the skull, and the eyes, as illustrated for example by the plane P in FIG. 3.

For this, the patient's posture can be stabilised by a gantry, for example installed along a wall. Alternatively or additionally, the horizontal position of the reference plane can be verified using a device 41 worn by the patient. As illustrated for example by FIG. 3, this device 41 can comprise glasses equipped with means for controlling the horizontal position of the reference plane. These means are for example mechanical means such as spirit levels, or electronic means such as an inertial measurement unit. A slot arranged on at least one arm of the glasses, in the longitudinal direction of the arm, can make it possible to verify the alignment of the patient's eye with the glasses. During the verification 180, the patient raises or lowers their head until the horizontal position is achieved. The horizontal position of the reference plane on the roll and pitch axes can be ensured by measuring the preferably vertical residual acceleration during the acquisition 18. Heading hold or yaw can be ensured by measuring the azimuth at the start of or before acquisition, this value remaining substantially constant during the acquisition 18 of the 3D scan.

The acquisition 18 can comprise an alert step when a modification of the patient's posture is observed, for example by the acceleration and azimuth measurements. The process 1 can furthermore comprise a verification of the 3D scan after the acquisition thereof, so as to reject an incorrectly positioned patient's scan, with for example the head at an angle or in which the posture changed during acquisition. The reliability of the process 1 is thus improved.

Homologous points 1100 are positioned 110 on the 3D scan. These homologous points enable a first processing of the scan, so as to remove scale effects between patients, to improve the alignment between the morphological data of different patients (processing commonly referred to as “registration”, aimed at aligning the same object on two different images using a conversion). Furthermore, these points can provide an anchor for determining the semi-homologous points, described hereinafter.

The homologous points 1100 can be positioned 110 manually by an operator, for example by a healthcare professional. This positioning 110 can be performed in an automated manner by the computer program product 2 or the remote server 3. After the automated positioning 110, the process 1 can comprise a verification of the operator of the position of these points, the operator being able to move them if their position is not correct.

At least three, preferably four, more preferably six, and even more preferably seven homologous points 1100 can be positioned 110 on the 3D scan. These points can be points located on the face, for example the base of both ears and/or the pupils, the nasal base, supplemented preferably by the tip of the chin. The points located on the face are easily identifiable on the 3D scan. When the homologous points 1100 are all points located on the patients face, the 3D scan only represent the patient's maxillofacial morphology.

Preferably, at least one, preferably two, and more preferably three additional homologous points 1100 are positioned 110 on the 3D scan, these points being disposed on the patient's chest, for example at the shoulder joints, and/or at the suprasternal notch. The 3D scan then represents the patient's maxillofacial and submandibular morphology. The process 1 thus makes it possible to account for not only the patient's maxillofacial morphology, but also the morphology of their chest. The morphological characteristics analysed represent a broader morphology of the patient. The process 1 is therefore improved.

Thanks to the homologous points 1100 on the patient's chest, determining the characteristic data of the patient's submandibular morphology is facilitated. Indeed, the submandibular morphology is essentially formed of soft tissue, for which the determination 11 of these data could induce a high variability impacting processing by the machine learning model, without the addition of these additional homologous points 1100. The reliability of the process is therefore improved further.

To facilitate the positioning 110 of these additional homologous points 1100, targets are preferably placed beforehand on the patient at these points, for example on both shoulder joints, and/or at the suprasternal notch, during the acquisition 18 of the 3D scan. The placing of these targets facilitates the identification of the homologous points 1100 of the chest regardless of the morphology of the patient's body, and particularly when the reliefs of the chest are not clearly visible on the 3D scan due to the presence of fat tissue.

The characteristic data of the patient's morphology are determined with respect to a reference morphology. The reference morphology can be a reference individual's morphology. A reference individual can be a patient chosen from the database. Preferably, the reference individual is chosen and/or determined from the patients of the database, for example the average shape of at least some, and preferably all, of the patients of the database, or the individual coming closest. The reference morphology can be obtained via an analysis configured to align the 3D scans in a single frame of reference via operations chosen from rotation, translation and scaling operations. According to an example, this analysis is a Procrustes analysis, preferably performed using homologous points 1100 positioned on the 3D scans of the database. This analysis is routinely used to compare the shapes after removing the components in rotation, translation and scaling. The reference morphology makes it possible to compile a reference grid (also known as “patch”) to be applied to the patient's 3D scan. The reference individual can be chosen when compiling the database, or preferably be updated when updating this database, described in detail hereinafter.

From the positioning 110 of the homologous points, the 3D scan can be processed so as to align the homologous points 1100 of the patient to those of the reference individual. For this, the reference grid can be applied on the 3D scan. The grid is then deformed to align the homologous points 1100 of the grid and those of the patient's 3D scan. Semi-homologous points 1110 can then be determined 111 to form characteristic data of the patient's morphology. It can be envisaged that these points be disposed on the grid, approximately equidistant from each other between the homologous points 1100, so as to map the surface of the 3D scan.

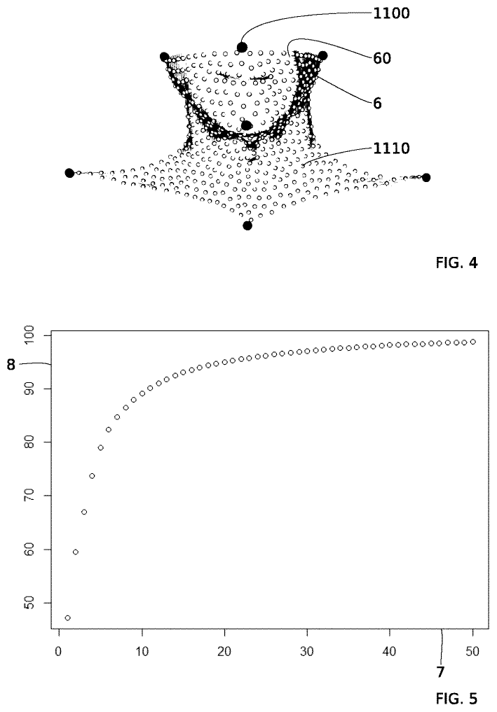

Preferably, the so-called sliding semi-homologous points, also known as sliding semi-landmarks, technique is used. This makes it possible to improve the biological correspondence of the semi-homologous points 1110 between patients, and therefore improve the reliability of the process 1 further. The semi-homologous points 1110 slide iteratively on the grid 60, as for example illustrated by FIG. 4. The iteration process is repeated until the deformation of the grid is minimised. The positioning of at least 200 and preferably at least 300 semi-homologous points can thus be optimised 111.

The homologous and/or semi-homologous points can then be replaced similarly to the patients from the database, so as to place all the individuals in the same space. For this, a second Procrustes analysis can be performed to align all of the homologous 1100 and/or semi-homologous points 1110 with all of the individuals of the database.

The characteristic data of the morphology of the patient 6 can comprise a set of homologous points 1100 and a set of semi-homologous points 1110 comprising at least 200 and preferably at least 300 points, these sets of points being disposed in a three-dimensional space, as for example illustrated by FIG. 4.

In order to limit the redundancy between the characteristic data of the morphology of different patients, the process can comprise a reduction 112 of the dimensionality of these data. This reduction 112 can be performed using any multidimensional analysis technique accessible to a person skilled in the art, according to the type of data (for example, qualitative or quantitative). Thus, the volume of data introduced into the machine learning model can be reduced, while retaining the essential part of the three-dimensional information of the patient's morphology.

According to an example, this reduction 112 can be performed using a principal component analysis (commonly abbreviated to PCA). The PCA has the effect of projecting the data into an orthogonal principal component base, in order to limit the number of variables required to capture the geometric variations between individuals, and only retain the data projected on this limited number of variables.

By way of example, FIG. 5 represents the proportion of variance expressed 8 by the characteristic morphology data represented, according to the number of principal components selected 7. By retaining the projected data into a base comprising the first four principal components, 75% of the variance is expressed, and 95% by retaining the data projected in a base comprising the first twenty principal components.

Preferably, the data projected in a base comprising at least the four, preferably at least the ten, and more preferably at least the twenty, first principal components are retained in the data set 12 which will be introduced 13 into the machine learning model.

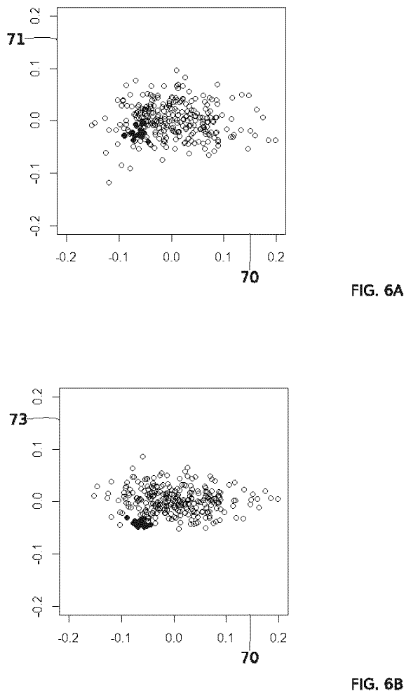

FIGS. 6A and 6B represent an example of projected data of a patient (in black) whose characteristic data of their morphology have been determined several times, using separate scans, and of all of the patients of the database (in white) in two planes defined by the first three principal components designated PC 1: 70, PC2: 71 and PC3: 73. The variability of the data of all of the patients is considerably greater than the variability of the data for a single patient whose analysis has been replicated. Any analytical error linked with the patient's posture, the acquisition by the 3D scanner, or the placement of the homologous points proves to be low compared to the natural variability of the data of all of the patients.

The data set 12 relating to the patient, introduced 13 into the machine learning model, can comprise the characteristic data of the patient's morphology, from the processing detailed above. Furthermore, this set 12 can comprise additional data 120. These data 120 can be the patient's clinical information data. Adding these data makes it possible to supplement the patient's morphological characteristics, and thus improve the process 1 further. For example, these data 120 can comprise information obtained via the medical questionnaire, such as the BERLIN and NOSAS questionnaires commonly used for the pre-diagnosis of OSAHS. Alternatively or additionally, these data 120 can comprise different morphological or physiological measurements such as the hip circumference, neck and waist circumferences, body mass index (BMI), calculated based on the patient's weight and height, arterial hypertension. Alternatively or additionally, these data 120 can comprise a representative criterion of the pharyngeal anatomy, for example qualified according to the Mallampati classification system. Note that the reduction of the dimensionality can be applied for the additional data 120.

Moreover, this set 12 can comprise additional characteristic data 121 of the patient's morphology in a prognathic position and/or a retrognathic position, these positions preferably maximum positions that can be achieved by the patient. For this, a 3D scan can be acquired for one or each of these positions. This or these 3D scan(s) can be processed in the manner detailed above for the 3D scan in the normal position. Adding these data makes it possible to account for the data of the cricomental space to obtain the apnoea+hypopnoea index, and thus improve the process 1 further. Furthermore, accounting for the cricomental space can make it possible to predict the success of a mandibular advancement device for the treatment of OSAHS.

The data set 12 relating to the patient is introduced 13 into the machine learning model. The machine learning module is trained on the database. The training consists of a learning phase, carried out on validated cases. The machine learning model can have been trained via several supervised classification procedures. In a non-limiting manner, mention can be made of different types of discriminant analyses, support vector machine, decision tree, neural networks, algorithms based on sets such as for example random forests (also known as decision tree forests), algorithms commonly named in the field as “Extreme gradient boosting”, abbreviated to XGBoost, “Adaptative Boosting”, abbreviated to ADABoost, “majority voting”, “soft voting”, or a combination thereof. The XGBoost algorithm particularly has the advantage of accepting the missing data in a data set. A person skilled in the art is capable to choosing the suitable learning procedure, particularly by assessing the predictive power of the model, similarly to the associated error. The trained machine learning model is configured to optimally predict an apnoea+hypopnoea index from the data set 12. The error associated with the predicted apnoea+hypopnoea index 14 for the data set 12, can be sent 160 and/or displayed 161 to the operator with the apnoea+hypopnoea index.

As mentioned above, the database comprises a plurality of patient data sets. Each data set can comprise morphology data of a patient from the set, the apnoea+hypopnoea index of the patient, supplemented by the additional 120 and supplementary data 121 detailed above. Preferably, at least a portion of the sets, and preferably each set, comprises the same data, from the same processing operations, as the data set 12 introduced 13 into the machine learning model. Note that the database is preferably anonymous, to meet the requirements relating to data protection.

According to an example, the database can comprise groups including several data sets of separate patients, grouped for example according to their anthropological type. Indeed, the reliability of the machine learning model prediction can be improved by comparing the morphological characteristics for a given anthropological type. For this, the data set 12 introduced 13 into the model can comprise a value, for example qualitative, of a representative class of the anthropological type, for example Caucasian, African, Asian.

If the patient whose apnoea+hypopnoea index is sought undergoes polygraphy or polysomnography, for example following the established diagnosis 15, the database can be updated 17. For this, the apnoea+hypopnoea index and the data set thereof can be sent 171 to the server 3 to be added to the database. The machine learning model can be reassessed on the new database, particularly to improve the predictive power thereof and minimise the prediction error. The model can thus itself be updated, to be used for future predictions. For example, the updated model can be sent from the remote server 3 to the computer program product 2. The database can preferably be updated with healthy patients, so as not to bias the model with the sole introduction of patients suffering from the condition.

A clinical study was carried out on a database of approximately 300 patients. This study demonstrated that there is a link between a patient's morphology and their predisposition to having OSAHS. From the patient's 3D scan, it was demonstrated that the process 1 makes it possible to obtain the apnoea+hypopnoea index with greater discrimination capabilities than those of the tools generally used for pre-diagnosis prior to polysomnography and ambulatory polygraphy, such as the NOSAS and BERLIN questionnaires.

In the light of the above description, it is clear that the invention proposes a solution for improving the reliability of and/or standardising, through the analysis of morphological characteristics of an upper part of the human body, the diagnosis of a condition, and more specifically the diagnosis of obstructive sleep apnoea-hypopnoea syndrome.

The invention is not limited to the aforementioned embodiments, and includes all the embodiments covered by the invention. The present invention is not limited to the examples described above. Many other alternative embodiments are possible, for example by combining features described above, without leaving the scope of the invention. Furthermore, the features described in relation to an aspect of the invention can be combined with another aspect of the invention.

1. A process for determining a patient's apnoea+hypopnoea index or a status dependent on an apnoea+hypopnoea index, comprising:
supplying a data set relating to a patient, to a remote server or a computer program product, the data set relating to the patient comprising, or making it possible to determine, characteristic data of the patient's at least maxillofacial morphology with respect to a reference morphology, said characteristic data being dependent on a positioning of at least four homologous points; on a 3D scan of a portion of the patient's head representing their at least maxillofacial morphology,
introducing the data set relating to the patient, comprising the characteristic data of the at least maxillofacial morphology of the patient, into a machine learning model trained to predict an apnoea+hypopnoea index; or a status dependent on an apnoea+hypopnoea index, for said data set, from a plurality of data sets of a database relating to a set of separate patients, each data set of the database:
comprising at least maxillofacial morphology data relating to a patient from the set, and
being associated with an apnoea+hypopnoea index, or a status dependent on an apnoea+hypopnoea index,
such that the learning model predicts an apnoea+hypopnoea index; or a status dependent on an apnoea+hypopnoea index, for the data set relating to the patient.

2. The process according to claim 1, wherein:
when the predicted apnoea+hypopnoea index for the data set relating to the patient is greater than 15, or
the status having at least one value associated with a low risk of having a condition and at least one value associated with an established risk of having the condition, when the predicted status has the at least one value associated with the established risk,
the process comprises a diagnosis of the patient's condition, for example obstructive sleep apnoea-hypopnoea syndrome.

3. The process according to claim 1, wherein:
when the predicted apnoea+hypopnoea index for the data set relating to the patient is between 15 and 30, or
the status having at least one value associated with a moderate established risk of having a condition and at least one value associated with a severe established risk of having a condition, when the predicted status has the at least one value associated with the moderate established risk,
the patient is diagnosed with a moderate degree of obstructive sleep apnoea-hypopnoea syndrome, and
when the predicted apnoea+hypopnoea index for the data set relating to the patient is greater than 30, or
when the predicted status has the at least one value associated with the severe established risk,
the patient is diagnosed with a severe degree of obstructive sleep apnoea-hypopnoea syndrome.

4. The process according to claim 1, wherein the supplied data set relating to the patient comprising a 3D scan of a portion of the patient's head representing the patient's at least maxillofacial morphology, the process comprises, prior to the introduction of the data set relating to the patient in the machine learning model:
positioning the at least one homologous points on the 3D scan,
determining, from the homologous points positioned, characteristic data of the patient's at least maxillofacial morphology with respect to the reference morphology.

5. The process according to claim 1, the supplied data set relating to the patient comprising a 3D scan of a portion of the patient's head, whereon the at least four homologous points are positioned, the process comprises, prior to the introduction of the data set relating to the patient in the machine learning model, determining, from the at least four homologous points positioned, characteristic data of the patient's at least maxillofacial morphology with respect to the reference morphology.

6. The process according to claim 1, wherein the supplied data set relating to the patient comprises characteristic data of the patient's at least maxillofacial morphology with respect to a reference morphology, as determined according to the positioning of the homologous points on the 3D scan.

7. The process according to claim 1, wherein the data set relating to the patient furthermore comprises, or makes it possible to furthermore determine, characteristic data of the patient's submandibular morphology with respect to the reference morphology, said characteristic data being dependent on a positioning of at least three supplementary homologous points, on the 3D scan of a portion of the patient's head representing their at least maxillofacial and submandibular morphology.

8. The process according to claim 1, wherein, the process comprising determining, from the homologous points positioned, characteristic data of the patient's at least maxillofacial morphology with respect to the reference morphology, determining the characteristic data of the patient's at least maxillofacial morphology with respect to the reference morphology comprises a reduction of the dimensionality of the data from positioning the at least four homologous points, for example by a principal component analysis.

9. The process according to claim 1, wherein the data set relating to the patient furthermore comprises additional clinical information data on the patient, for example chosen from a patient body mass index, clinical examination upper airway obstruction indexes, medical questionnaire data, the machine learning model being trained to predict the apnoea+hypopnoea index; or the status dependent on an apnoea+hypopnoea index, for the data set relating to the patient, from the plurality of data sets of the database, each data set of the database comprising said additional clinical information data relating to a patient of the set.

10. The process according to claim 1, wherein the data set relating to the patient furthermore comprises, or makes it possible to furthermore determine, supplementary characteristic data of the patient's maxillofacial morphology in at least one position from a prognathic position and a retrognathic position.

11. The process according to claim 1, the process comprising the acquisition of the 3D scan of a portion of the patient's head representing their at least maxillofacial morphology, the acquisition of the 3D scan comprising a verification of the alignment of the patient's head, by an alignment device.

12. The process according to claim 1, wherein, when the data set relating to the patient is supplied to the remote server, the process comprises returning, from the remote server to a computer program product, the apnoea+hypopnoea index, or the status, predicted for the data set relating to the patient.

13. The process according to claim 1, wherein, when the data set relating to the patient is supplied to the computer program product or when a computer program product receives the apnoea+hypopnoea index, or the status, returned from the remote server, the process comprises displaying, by the computer program product, the apnoea+hypopnoea index, or the status, predicted for the data set relating to the patient.

14. A remote server capable of communicating with a computer program product, for the implementation of the process according to claim 1, comprising:
a database relating to a set of distinct patients comprising a plurality of data sets, each data set of the database:
comprising at least maxillofacial morphology data relating to a patient from the set,
being associated with an apnoea+hypopnoea index, or a status dependent on an apnoea+hypopnoea index, and
a machine learning model trained to predict an apnoea+hypopnoea index, or a status dependent on an apnoea+hypopnoea index, for said data set, from the database,
the remote server being configured to:
receive the data set relating to the patient comprising, or making it possible to determine, characteristic data of the patient's at least maxillofacial morphology with respect to a reference morphology, said data being dependent on the positioning of at least four homologous points, on a 3D scan of a portion of the patient's head representing their at least maxillofacial morphology,
introduce the data set relating to the patient, comprising the characteristics of the patient's at least maxillofacial morphology, in the trained machine learning model, such that the learning model predicts an apnoea+hypopnoea index or a status dependent on an apnoea+hypopnoea index, for the data set relating to the patient.

15. The remote server according to claim 14, the remote server being configured to update the database following a receipt of a dataset relating to the patient, supplemented by the patient's apnoea+hypopnoea index measured by polysomnography or ambulatory polygraphy.

16. A computer program product stored on a non-transitory computer-readable medium and for implementing the process according to claim 1, comprising instructions, which when they are performed by at least one processor, which executes at least, sending to a remote server the data set relating to the patient, the data set relating to the patient comprising, or making it possible to determine, characteristic data of the patient's at least maxillofacial morphology with respect to a reference morphology, said data being dependent on the positioning of at least four homologous points, on a 3D scan of a portion of the patient's head representing their at least maxillofacial morphology.

17. A computer program product stored on a non-transitory computer-readable medium and for implementing the process according to claim 1, comprising instructions, which when they are performed by at least one processor, which executes at least:
receiving the data set relating to the patient comprising, or making it possible to determine, characteristic data of the patient's at least maxillofacial morphology with respect to a reference morphology, said data being dependent on the positioning of at least four homologous points, on a 3D scan of a portion of the patient's head representing their at least maxillofacial morphology,
introducing the data set relating to the patient, comprising the characteristic data of the at least maxillofacial morphology of the patient, into the machine learning model trained to predict an apnoea+hypopnoea index, or a status dependent on an apnoea+hypopnoea index, for said data set, from a plurality of data sets of a database relating to a set of separate patients, each data set of the database:
comprising at least maxillofacial morphology data relating to a patient from the set, and
being associated with an apnoea+hypopnoea index, or a status dependent on an apnoea+hypopnoea index,
such that the learning model predicts an apnoea+hypopnoea index, or a status dependent on an apnoea+hypopnoea index, for the data set relating to the patient.

18. A kit for implementing the process according to claim 1, comprising at least one computer program product stored on a non-transitory computer-readable medium and comprising instructions, which when executed by at least one processor, which execute at least:
sending to a remote server the data set relating to the patient, the data set relating to the patient comprising, or making it possible to determine, characteristic data of the patient's at least maxillofacial morphology with respect to a reference morphology, said data being dependent on the positioning of at least four homologous points, on a 3D scan of a portion of the patient's head representing their at least maxillofacial morphology; or
receiving the data set relating to the patient comprising, or making it possible to determine, characteristic data of the patient's at least maxillofacial morphology with respect to a reference morphology, said data being dependent on the positioning of at least four homologous points, on a 3D scan of a portion of the patient's head representing their at least maxillofacial morphology,
introducing (13) the data set (12) relating to the patient, comprising the characteristic data of the at least maxillofacial morphology of the patient, into the machine learning model trained to predict an apnoea+hypopnoea index, or a status dependent on an apnoea+hypopnoea index, for said data set, from a plurality of data sets of a database relating to a set of separate patients, each data set of the database:
comprising at least maxillofacial morphology data relating to a patient from the set, and
being associated with an apnoea+hypopnoea index, or a status dependent on an apnoea+hypopnoea index,
such that the learning model predicts (14) an apnoea+hypopnoea index, or a status dependent on an apnoea+hypopnoea index, for the data set relating to the patient; and
a scanner capable of the acquisition of a 3D scan of a portion of the patient's head representing their at least maxillofacial morphology.

19. The kit according to the claim 18, furthermore comprising a device (41) for aligning the patient's head.
